(19) United States
(12) Patent Application Publication
(10) Pub. No.: US 2008/0070855 A1
(43) Pub. Date: Mar. 20, 2008

(54) Treatment with anti-VEGF agents to prevent postoperative inflammation and angiogenesis in normal and diseased eyes

(76) Inventor: James Pitzer Gills, Palm Harbor, FL (US)

Correspondence Address: KRAMER & AMADO, P.C., 1725 DUKE STREET, SUITE 240, ALEXANDRIA, VA, 22314, US

(21) Appl. No.: 11/523,563

(22) Filed: Sep. 20, 2006

(51) Int. Cl. A61K 48/00; A61K 38/17; A61K 31/506; A61K 39/395

(52) U.S. Cl. 514/44; 514/12; 424/145.1; 514/252.18

(57) ABSTRACT

Provided are methods for preventing post-operative neovascularization and/or inflammation in ophthalmic tissue of a patient undergoing ophthalmic surgery by administering a therapeutically effective amount of an anti-VEGF compound to the ophthalmic tissue of a patient where the anti-VEGF compound is administered to the patient prior to the time the ophthalmic surgery is performed on the patient to one hour following the time the ophthalmic surgery is performed on the patient.

### BACKGROUND

Intraocular surgeries result in the release of a protein called vascular endothelial growth factor (VEGF) which leads to an inflammatory response. This response has both desirable effects that promote healing, and undesirable effects that can delay recovery. In some patients, this response may lead to the proliferation of abnormal vascular growth, or angiogenesis. Patients who have certain eye diseases such as wet macular degeneration and diabetic retinopathy are at particular risk of a more severe angiogenic response.

Both intraocular and extraocular surgeries may lead to inflammation of the ophthalmic tissue and furthermore may cause neovascularization during the healing process. Some examples of these are: lid procedures, pterygium removal, full or partial thickness corneal transplant, glaucoma filtration procedures, cataract extraction and intraocular lens implantation, retinal detachment repair, and vitrectomy. Intraocular procedures in particular can cause an angiogenic response that is especially harmful if the patient has pre-existing disease. In these cases, the procedure further exacerbates the problem by increasing the neovascularization and causing the disease process to progress.

Cataract extraction and intraocular lens implantation is one of the most commonly performed intraocular procedures, and may be performed in various fashions. Regardless of the technique used to remove the cataract, the healing time from the procedure is typically delayed for patients with pre-existing conditions such as: wet macular degeneration, diabetic retinopathy and conditions secondary to diabetic retinopathy, and vascular occlusive disease.

To prevent inflammation following intraocular surgery, topical anti-inflammatory medications, including steroids and/or non-steroidal anti-inflammatory drugs (NSAIDs) are typically administered. Intraocular surgery may result in neovascularization whereby new blood vessels are formed in the eye thereby inhibiting or delaying the recovery by the patient. Additional complications of cataract surgery include, but are not limited to retinal detachment, cystoid macular degeneration, bullous keratopathy, choroidal hemorrhage, endophthalmitis, posterior capsular opacification and glaucoma.

Two diseases that particularly predispose patients to a more severe angiogenic response following surgery are age-related macular degeneration and diabetic retinopathy.

Age-related macular degeneration (ARMD) is a degenerative condition of the macula (the central retina). Macular degeneration is the most common cause of vision loss in the United States in those 50 or older, and its prevalence increases with age. There are two different forms of macular degeneration, dry form and wet form. In dry macular degeneration (atrophic macular degeneration), there is pigmentary disturbance in the macular region but no elevated macular scarring and no hemorrhage or exudation in the region of the macula. In wet macular degeneration (exudative macular degeneration) a subretinal network of choroidal neovascularization is formed by the body to try to provide oxygen to the retina, leading to intraretinal hemorrhage, subretinal fluid, pigment epithelial detachment and hyperpigmentation. Eventually, the neovascularization complex contracts and leaves a distinct elevated scar at the posterior pole.

There is no proven surgical therapy for dry macular degeneration. In selected cases of wet macular degeneration, laser photocoagulation is effective for sealing leaking or bleeding vessels. Unfortunately, laser photocoagulation usually does not restore lost vision, but it may prevent further loss. Photodynamic therapy has proven to be effective in stopping abnormal blood vessel growth in some patients with wet AMD. This type of laser treatment is far less damaging than laser photocoagulation and is the treatment of choice in many cases. As with all intraocular surgical procedures, including those conducted solely with the use of a laser, the surgical patient may suffer from intraocular inflammation and possible neovascularization. To prevent inflammation, the postoperative patient may be prescribed topical anti-inflammatory medications, such as steroids and/or NSAIDs.

Diabetic retinopathy is a major cause of blindness and is particularly severe in patients who have insulin-dependent diabetes mellitus (IDDM; type I diabetes mellitus) and patients with chronic non-insulin-dependent diabetes mellitus (NIDDM; type II diabetes mellitus). Over time, diabetes affects the circulatory system of the retina. The earliest phase of the disease is known as background diabetic retinopathy. In this phase, the arteries in the retina become weakened and leak, forming small, dot-like hemorrhages. These leaking vessels often lead to swelling or edema in the retina and decreased vision.

The next stage is known as proliferative diabetic retinopathy. In this stage, circulation problems cause areas of the retina to become oxygen-deprived or ischemic. New, fragile vessels develop as the circulatory system attempts to maintain adequate oxygen levels within the retina. This process is called neovascularization. The delicate vessels hemorrhage easily leading to vitreous hemorrhages. Blood may leak into the retina and vitreous, causing symptoms of spots or floaters, along with decreased vision. In the later phases of the disease, continued abnormal vessel growth and scar tissue may cause serious problems such as retinal detachment, rubeosis of the iris and glaucoma.

Diabetic retinopathy is treated in many ways depending on the stage of the disease and the specific problem that requires attention. The retinal surgeon relies on several tests to monitor the progression of the disease and to make decisions for the appropriate treatment. These include fluorescein angiography, optical coherence tomography, and ultrasound imaging of the eye.

The abnormal growth of tiny blood vessels and the associated complication of bleeding is one of the most common problems treated by vitreo-retinal surgeons. Laser surgery called pan retinal photocoagulation (PRP) is usually the treatment of choice for this problem. PRP may diminish or eliminate proliferative retinopathy and neovascularization of the iris. With PRP, the surgeon uses laser to destroy oxygen-deprived retinal tissue outside of the patient's central vision. While this creates blind spots in the peripheral vision, PRP prevents the continued growth of the fragile vessels and seals the leaking ones. The goal of the treatment is to arrest the progression of the disease.

Vitrectomy is another surgery commonly needed for diabetic patients who suffer a vitreous hemorrhage (bleeding in the gel-like substance that fills the center of the eye). During a vitrectomy, the retinal surgeon carefully removes blood and vitreous from the eye, and replaces it with clear salt solution (saline). At the same time, the surgeon may also gently cut strands of vitreous attached to the retina that create traction and could lead to retinal detachment or tears.

Patients with diabetes are at greater risk of developing retinal tears and detachment. Tears are often sealed with laser surgery. Retinal detachment requires surgical treatment to reattach the retina to the back of the eye. The prognosis for visual recovery is dependent on the location and severity of the detachment.

Following both PRP and vitrectomy, recovering patients can develop inflammation of the intraocular tissue. Further complications may include neovascularization as the intraocular tissue heals from the surgery. To reduce intraocular inflammation and the possibility of infection, surgical patients are often prescribed with oral and/or topical anti-inflammatory medications such as corticosteroids and NSAIDs and/or antibiotics.

### SUMMARY

Various exemplary embodiments relate to a method for preventing post-operative neovascularization in ophthalmic tissue of a patient undergoing ophthalmic surgery by administering an effective amount of an anti-VEGF compound to the ophthalmic tissue of a patient. In various exemplary embodiments, the anti-VEGF compound is administered to the patient prior to the time the ophthalmic surgery is performed on the patient to one hour following the time the ophthalmic surgery is performed on the patient.

Various exemplary embodiments relate to a method for preventing post-operative inflammation in ophthalmic tissue of a patient undergoing ophthalmic surgery by administering an effective amount of an anti-VEGF compound to the ophthalmic tissue of a patient. In various exemplary embodiments, the anti-VEGF compound is administered to the patient prior to the time the ophthalmic surgery is performed on the patient to one hour following the time the ophthalmic surgery is performed on the patient.

Various exemplary embodiments relate to a method for preventing post-operative neovascularization and post-operative inflammation in ophthalmic tissue of a patient undergoing ophthalmic surgery by administering an effective amount of an anti-VEGF compound to the ophthalmic tissue of a patient. In various exemplary embodiments, the anti-VEGF compound is administered to the patient prior to the time the ophthalmic surgery is performed on the patient to one hour following the time the ophthalmic surgery is performed on the patient.

Various exemplary embodiments include a method for administering an anti-VEGF compound from fourteen days prior to the time the ophthalmic surgery is performed on the patient to one hour prior to the time the ophthalmic surgery is performed on the patient.

Various exemplary embodiments include a method for administering an anti-VEGF compound from seven days prior to the time the ophthalmic surgery is performed on the patient to one day prior to the time the ophthalmic surgery is performed on the patient.

Various exemplary embodiments include a method for administering an anti-VEGF compound such as of bevacizumab, pegaptanib sodium, ranibizumab, VEGF-Traps and/or siRNAs.

Various exemplary embodiments include a method for administering an anti-VEGF compound such as bevacizumab, pegaptanib sodium and/or ranibizumab.

In various exemplary embodiments ophthalmic surgery includes intraocular and/or extraocular surgery.

In various exemplary embodiments extraocular and intraocular surgery include: pterygium removal, lid procedures, cataract surgery, glaucoma surgery, retinal surgery, or corneal transplant surgery.

### DETAILED DESCRIPTION

For simplicity and illustrative purposes, the principles are shown by way of examples of compositions and methods described. In the following description, numerous specific details are set forth in order to provide a thorough understanding of the examples. It will be apparent however, to one of ordinary skill in the art, that the examples may be practiced without limitation to these specific details. In other instances, well known methods and compositions are not described in detail so as not to unnecessarily obscure understanding of the examples.

Vascular endothelial growth factor (hereinafter referred to as VEGF, also known as VEGF-A) is the primary factor in the formation of new blood vessels or angiogenesis in the body. In particular, VEGF stimulates vascular endothelial cell growth, survival, and proliferation. VEGF promotes angiogenesis in normal tissue and neovascularization as well. Neovascularization is defined as proliferation of blood vessels in tissue not normally containing them, or proliferation of blood vessels of a different character than are usually found in a specific tissue. As applied to a preventative method, neovascularization particularly relates to the production and/or proliferation of new or different blood vessels in the intraocular region.

VEGF is a product of inflammation and has been linked to tissue remodeling throughout the body. While the exact mechanism is unknown, tissues upgrade inflammation by increasing the oxidative process resulting in the upregulation of the VEGF receptor on the cell surface. The upregulation of VEGF receptors results in the production, transport to and action of VEGF on the cell membrane. The mechanism may function through nitrous dependent pathways. There are several other mechanisms of inflammation that may result in the upregulation of VEGF. General inflammation in response to cellular injury, marked by inflammatory conditions, may be instigated by VEGF as well as take part in the VEGF response. Without being bound to a particular theory, various embodiments of the invention include methods for preventing postoperative inflammation and/or angiogensis in ophthalmic tissue injured during surgical procedures by selectively targeting VEGF but allowing other inflammatory or anti-inflammatory mechanisms that are beneficial to the healing process.

Inflammation generally refers to the local response to cellular injury that is marked by capillary dilatation, leukocytic infiltration, redness, heat, pain, and swelling. As applied to a preventative method, inflammation particularly relates to the cellular injury within the eye region which may be caused by injury or disease, or by surgery of the ophthalmic tissue, particularly intraocular surgery.

The term ophthalmic tissue is defined as any tissue generally associated with the eye. The ophthalmic tissue may include, but is not limited to cornea, conjunctive, sclera, aqueous humor, vitreous humor, iris, lens, globe, retina, macula, eyelids and other tissues or cells that are known in the art to make up the structures or components of the eye. Ophthalmic surgery is defined as any surgery or invasive therapy directed upon the aforementioned tissues or components of the eye. Intraocular surgery is defined as surgery that takes place within the eye itself.

Anti-VEGF compounds are compounds that target VEGF and/or the VEGF pathway and inhibit the inflammatory and/or angiogenic function of the growth factor using various mechanisms. Anti-VEGF compounds may function by blocking the product, transfer and/or action of VEGF. Anti-VEGF compounds may reduce the action of VEGF at any point in the VEGF pathway. Particular functions for anti-VEGF compounds may include, but are not limited to, regulating the genes that encode the production or regulation of VEGF, preventing the transcription of sequences that transcribe or induce the production of VEGF, binding to or otherwise inhibiting transcribed RNA required for the production of VEGF, directly binding to VEGF to prevent its binding to cells or receptors, binding to VEGF to prevent its release or transfer from cells, binding to VEGF to induce destruction of the growth factor and blocking the receptors that bind VEGF.

Various exemplary embodiments of anti-VEGF compounds include pharmaceutical agents that show efficacy in inhibiting pre-retinal neovascularization including, but are not limited to, agents that block VEGF expression or neutralize VEGF once expressed, antibodies against VEGF, VEGF-neutralizing chimeric proteins, VEGF antagonists and VEGF receptor blockers.

Anti-VEGF treatments have been administered for the prevention of macular degeneration as an alternative to photocoagulation. In particular, the FDA has approved two different methods for administering anti-VEGF compounds for treating wet macular degeneration. One such method is in the intravitreal injection of ranibizumab. Ranibizumab (trade name LUCENTIS™ available from Genentech) is a recombinant, humanized monoclonal Fab fragment designed to actively bind and inhibit VEGF. The FDA has approved the intravitreal injection of 2.5 mg of ranibizumab for the treatment of wet AMD.

A second approved anti-VEGF treatment for AMD is pegaptanib sodium. Pegaptanib sodium (trade name MACUGEN® available from Pfizer, Inc.) is a pegylated aptamer which is conjugated to polyethylene glycol. The conjugated oligonucleotide is designed to specifically bind and neutralize VEGF₁₆₅ which has been targeted as the predominant isomer in human choroidal neovascularization.

Bevacizumab (trade name AVASTIN® available from Genentech) is another anti-VEGF humanized monoclonal IgG1 antibody that binds and inhibits VEGF. Bevacizumab is currently being tested for efficacy in AMD trials.

Additional anti-VEGF treatments include the VEGF Trap which is a high-affinity recombinant fusion protein which consists of the immunoglobulin domain 2 of the VEGF-R1 receptor and the domain 3 of the VEGF-R2 receptor fused to the crystallizable fragment of human IgG. The VEGF Trap selectively binds and neutralizes all VEGF isoforms. Transcriptional related anti-VEGF agents may include the use of small interfering RNA (siRNA) which targets and destroys mRNAs, thereby silencing the expression of the target gene. Two siRNAs that specifically target VEGF are currently being investigated for the treatment of macular degeneration, including Cand5 (Acuity Pharmaceuticals, Philadelphia, Pa.) and Sima-027 (Sirna Therapeutics, Boulder, Colo.).

Various exemplary embodiments of anti-VEGF agents include, but are not limited to, VEGF inhibitors currently used in cancer trials such as PTK787, which is an receptor tyrosine kinase (“RTK”) inhibitor from Novartis; Bay 43-9006, which is a RAF kinase inhibitor from Bayer/Onyx; SU1248 and AG 013676, which are RTK inhibitors from Pfizer; ZD 6474 which is an RTK inhibitor from AstraZeneca; and VEGF-trap soluble receptor from Regeneron.

The term anti-VEGF compound encompasses all of the compounds and therapies listed above and any future therapies that in some manner inhibit or interfere with the function of VEGF either in contributing to neovascularization and/or in contributing to inflammation.

In various exemplary embodiments, at least one anti-VEGF compound is formulated in a pharmaceutical composition for delivery to a subject. The subject may include a mammal, particularly, but not limited to, a human. In various exemplary embodiments, the pharmaceutical composition is administered systemically or locally. Methods of administration include oral, topical, parenteral delivery or injection directly into the area of surgery. Injectable pharmaceutical compositions may include fillers, diluents, carriers and/or other inert compounds for effecting delivery of the anti-VEGF compound as are well known in the art.

In various exemplary embodiments, a pharmaceutical composition contains a therapeutically effective amount of the anti-VEGF compound for systemic or local administration. The term therapeutically effective amount is the amount of anti-VEGF compound required to provide an inhibiting or alleviating effect with regards to neovascularization and/or inflammation. A therapeutically effective amount of a pharmaceutical composition is the amount of composition required to provide a therapeutically effective amount of anti-VEGF compound.

In various exemplary embodiments, the specific anti-VEGF compounds cited above are used in the following ranges of therapeutically effective amounts:

In various exemplary embodiments, the therapeutically effective amount of bevacizumab ranges from 0.01 mg to 5 mg of active agent within the pharmaceutical composition. Pharmaceutical compositions for delivering the anti-VEGF agent may range from solutions containing 0.0004 cc to 0.2 cc bevacizumab. In various exemplary embodiments, the therapeutically effective amount of pegaptanib sodium ranges from 0.001 mg to 3 mg within the pharmaceutical composition. In various exemplary embodiments, the therapeutically effective amount of ranibizumab ranges from 0.01 mg to 6 mg of active agent.

In various exemplary embodiments pharmaceutical compositions for local delivery by injection include carriers for preparing a solution containing an anti-VEGF compound. Such carriers or diluents may be particularly suited to maintain the stability of the anti-VEGF compound. Carriers and diluents may also be chosen to be suitable for the location of delivery, for example into the eye. Such carriers and diluents are well known in the art as many of the anti-VEGF compounds are provided in solution for injection.

In various exemplary embodiments, the pharmaceutical compositions for local delivery may be injected directly into the ophthalmic tissue. An injection into the ophthalmic tissue may include, but is not limited to injection into the cornea, conjunctive, sclera, aqueous humor, vitreous humor, iris, lens, globe, retina, macula and other tissues or cells that are known in the art to make up the structures or components of the eye.

In various exemplary embodiments, the preventative method includes the injection of a therapeutically effective amount of an anti-VEGF compound into the vitreous humor of the eye. Injection into the vitreous humor is known in the art as intravitreal injection. In a method for intravitreal injection, the anti-VEGF compound is dosed as an injection using a needle to pierce the sclera into the vitreous. In various exemplary embodiments, the anti-VEGF compound may be injected into the sclera as part of a pharmaceutical composition.

In various exemplary embodiments, a preventative method is performed by administering an anti-VEGF compound to prevent neovascularization and/or inflammation of the ophthalmic tissue following ophthalmic surgery. The time period following ophthalmic surgery is defined as the post-operative period. The condition of the patient following ophthalmic surgery is defined as the post-operative condition. The time period prior to the ophthalmic surgery is defined as the pre-operative period. The condition of the patient before ophthalmic surgery is defined as the pre-operative condition.

In various exemplary embodiments, anti-VEGF compounds are used alone or in combination with other steroids and non-steroidal anti-inflammatory drugs to reduce inflammation. In various embodiments, the anti-VEGF compounds, alone or in combination with other active agents are give hours to weeks prior to surgery depending on the problem.

Ophthalmic surgery is defined as any surgery associated with ophthalmic tissue. In various exemplary embodiments, the ophthalmic surgery is intraocular surgery such as, but not limited to, cataract surgery, glaucoma surgery, macular degeneration surgery and vascular retinopathy surgery.

In various exemplary embodiments, the ophthalmic surgery is extraocular surgery such as, but not limited to, various eyelid procedures, and pterygium.

In various exemplary embodiments, the timing of administration of the anti-VEGF compound depends on the timing of the ophthalmic surgery. The time for performing ophthalmic surgery is defined as beginning with the initiation of surgery and ending with the completion of the surgical procedure. In various exemplary embodiments, a preventative method includes administering an anti-VEGF compound at any time prior to the time for performing ophthalmic surgery to one hour following the time for performing ophthalmic surgery. In various exemplary embodiments, a preventative method includes administering an anti-VEGF compound from fourteen days prior to ophthalmic surgery to one hour prior to ophthalmic surgery to. In various exemplary embodiments, a preventative method includes administering an anti-VEGF compound from seven days prior to ophthalmic surgery to one day prior to ophthalmic surgery. The administration of the anti-VEGF compound may be performed in one or more multiple doses throughout the time frames of the various embodiments.

The method for preventing neovascularization and inflammation reduces healing time for patients with pre-existing conditions following intraocular surgeries. In various embodiments, pre-existing conditions that are improved as a result of injecting anti-VEGF agents at the time before and during intraocular surgery include, but are not limited to, cystoid macular edema, diabetic retinopathy and related conditions such as rubeosis of the iris, vitreous hemorrhage, retinal vascular occlusive disease and retinopathy of prematurity (ROP). In various embodiments, the method for administering anti-VEGF compounds includes treating pterygiums and minimizing inflammation associated with oculoplastic surgery.

The method for administering an anti-VEGF compound, in a given time frame prevents neovascularization and inflammation of the ocular tissue following intraocular and/or extraocular surgery. The method has been found to be effective in obliterating pterygiums and other types of surface neovascularization. In various embodiments, the method obviates the need for treatment with postoperative anti-inflammatory drugs and/or antibiotics. Patients treated by the method showed decreased complications including decreased neovascularization and decreased inflammation as described in the examples below.

### EXAMPLES

### Example 1

### Method for Intravitreal Injection

The following is an example of a method performed for the intravitreal injection of pharmaceutical compositions containing at least one anti-VEGF compound. Prior to injecting a needle through the sclera, the eyes were massaged to soften the ophthalmic tissue. The next step is to create a bleb in the conjunctiva and move the conjunctiva up from prefere so it covered an area about 3.5 mm from the limbus. Once the conjunctiva is prepared, the needle is inserted through the sclera into the vitreous. A 30 or 31 gauge needle is used. Once the needle is in the vitreous, the pharmaceutical composition containing a therapeutically effective amount of the anti-VEGF compound is injected pars plana into the vitreous.

### Example 2

A 67 year old male underwent cataract surgery in the right eye. The patient suffered from diabetic retinopathy in the right eye and was pseudophakic in the left eye. Refraction of the right eye was measured as −0.25−0.75×170 20/200. Bevacizumab (AVASTIN® available from Genentech) was injected intravitreally into the right eye according to the method of Example 1. The pharmaceutical composition contained 0.15 cc (3.75 mg) of bevacizumab. The injection was performed prior to the cataract surgery on the same day as the surgery. Postoperative medications prescribed included prednisolone twice a day applied to the right eye and gatifloxacin applied once daily to the right eye. After one week the patient was doing well. Refraction of the right eye after one week was measured at −0.25 sph 20/25.

### Example 3

A 65 year old female with proliferative diabetic retinopathy with a history of pan-photocoagulation underwent cataract surgery of both eyes, however on different dates. Preoperative right eye refraction was measured as pl −0.50×174 20/100. Preoperative left eye refraction was measured as −0.75 sph 20/150. Bevacizumab (AVASTIN® available from Genentech) was injected into both eyes intravitreally according to the method of Example 1. The pharmaceutical composition administered to each eye contained 0.1 cc (2.5 mg) of bevacizumab. The bevacizumab was injected prior to surgery on the same day that cataract surgery was performed on the right eye. Post-operative medications for the right eye included prednisolone applied topically once a day and ketorolac applied topically once a day. Nine days after bevacizumab injection, cataract surgery was performed on the left eye. No postoperative medications were prescribed for the left eye. In an examination performed one month following cataract surgery of the right eye, refraction of the right eye was +0.50 sph 20/20. Refraction of the left eye was plano sph 20/40.

### Example 4

A 58 year old female with proliferative diabetic retinopathy with a history of pan-photocoagulation underwent cataract surgery of the left eye. Preoperative left eye refraction was measured as plano sph 20/80. Bevacizumab (AVASTIN® available from Genentech) was injected into the left eye intravitreally according to the method of Example 1. The pharmaceutical composition administered to the left eye contained 0.1 cc (2.5 mg) of bevacizumab. The bevacizumab was injected one week prior to cataract surgery. Postoperative medications prescribed following cataract surgery included prednisolone twice a day and ketorolac applied topically twice a day to the left eye. Six days following cataract surgery refraction of the left eye was measured as plano sph 20/25.

What is claimed is:

1. A method for preventing post-operative neovascularization in ophthalmic tissue of a patient undergoing ophthalmic surgery comprising:
administering a therapeutically effective amount of an anti-VEGF compound to the ophthalmic tissue of a patient, wherein ophthalmic surgery is performed on the patient during a period of time, wherein the anti-VEGF compound is administered to the patient prior to the time the ophthalmic surgery is performed on the patient to one hour following the time the ophthalmic surgery is performed on the patient.

2. The method according claim 1, wherein the anti-VEGF compound is administered from fourteen days prior to the time the ophthalmic surgery is performed on the patient to one hour prior to the time the ophthalmic surgery is performed on the patient.

3. The method according to claim 2, wherein the anti-VEGF compound is administered from seven days prior to the time the ophthalmic surgery is performed on the patient to one day prior to the time the ophthalmic surgery is performed on the patient.

4. The method according to claim 1, wherein the anti-VEGF compound is selected from the group consisting of bevacizumab, pegaptanib sodium, ranibizumab, a VEGF-Trap and a siRNA.

5. The method according to claim 4, wherein the anti-VEGF compound is selected from the group consisting of bevacizumab, pegaptanib sodium and ranibizumab.

6. The method according to claim 1, wherein the ophthalmic surgery is intraocular surgery.

7. The method according to claim 6, wherein the intraocular surgery is selected from the group consisting of cataract surgery, corneal surgery, glaucoma surgery, retina surgery and vascular retinopathy surgery.

8. A method for preventing post-operative inflammation in ophthalmic tissue of a patient undergoing ophthalmic surgery comprising:
administering a therapeutically effective amount of an anti-VEGF compound to the ophthalmic tissue of a patient, wherein ophthalmic surgery is performed on the patient during a period of time, wherein the anti-VEGF compound is administered to the patient prior to the time the ophthalmic surgery is performed on the patient to one hour following the time the ophthalmic surgery is performed on the patient.

9. The method according claim 9, wherein the anti-VEGF compound is administered from fourteen days prior to the time the ophthalmic surgery is performed on the patient to one hour prior to the time the ophthalmic surgery is performed on the patient.

10. The method according to claim 10, wherein the anti-VEGF compound is administered from seven days prior to the time the ophthalmic surgery is performed on the patient to one day prior to the time the ophthalmic surgery is performed on the patient.

11. The method according to claim 9, wherein the anti-VEGF compound is selected from the group consisting of bevacizumab, pegaptanib sodium, ranibizumab, a VEGF-Trap and a siRNA.

12. The method according to claim 12, wherein the anti-VEGF compound is selected from the group consisting of bevacizumab, pegaptanib sodium and ranibizumab.

13. The method according to claim 9, wherein the ophthalmic surgery is intraocular surgery.

14. The method according to claim 13, wherein the intraocular surgery is selected from the group consisting of cataract surgery, glaucoma surgery, macular degeneration surgery and vascular retinopathy surgery.

15. A method for preventing post-operative neovascularization and post-operative inflammation in ophthalmic tissue of a patient undergoing ophthalmic surgery comprising:
administering a therapeutically effective amount of an anti-VEGF compound to the ophthalmic tissue of a patient, wherein ophthalmic surgery is performed on the patient during a period of time, wherein the anti-VEGF compound is administered to the patient prior to the time the ophthalmic surgery is performed on the patient to one hour following the time the ophthalmic surgery is performed on the patient.

16. The method according claim 15 wherein the anti-VEGF compound is administered from fourteen days prior to the time the ophthalmic surgery is performed on the patient to one hour prior to the time the ophthalmic surgery is performed on the patient.

17. The method according to claim 16 wherein the anti-VEGF compound is administered from seven days prior to the time the ophthalmic surgery is performed on the patient to one day prior to the time the ophthalmic surgery is performed on the patient.
